(19) United States
(12) Patent Application Publication
(10) Pub. No.: US 2026/0355099 A1
(43) Pub. Date: Nov. 12, 2026

(54) COLOR COSMETICS FORMULATION

(71) Applicant: Coty Inc., New York, NY (US)

(72) Inventors: Nicholas Quinteros, New York, NY (US); Camille Le Jolu, New York, NY (US)

(21) Appl. No.: 19/714,356

(22) Filed: Jun. 19, 2026

Related U.S. Application Data
(63) Continuation of application No. PCT/US2025/010242, filed on Jan. 3, 2025.
(60) Provisional application No. 63/618,163, filed on Jan. 5, 2024.

(51) Int. Cl. A61K 8/891; A61K 8/06; A61K 8/25; A61K 8/58; A61K 8/87; A61Q 19/00

(52) U.S. Cl. CPC A61K 8/891; A61K 8/064; A61K 8/25; A61K 8/585; A61K 8/87; A61Q 19/00; A61K 2800/10; A61K 2800/95

(57) ABSTRACT

Various embodiments disclosed relate to a cosmetic formulation. A formulation may include a discontinuous water phase. A formulation may include a continuous oil phase comprising: a film former including an MQ resin, an emollient, an emulsifier; and a thickener, wherein the formulation subsequent to application to a surface demonstrates wear of at least 24 hours, and wherein the formulation subsequent to application to the surface demonstrates a tack of at least 150 at half an hour subsequent to application to the surface.

### CLAIM OF PRIORITY

This patent application is a continuation of PCT Application Serial No. PCT/US2025/010242, filed Jan. 3, 2025, which application claims the benefit of priority to U.S. Application Ser. No. 63/618,163, filed Jan. 5, 2024, which are incorporated by reference herein in their entireties.

### BACKGROUND

Various cosmetic formulations are used by consumers to cover perceived imperfections, apply a desired color, or another purpose. For a product to be viable for consumer use, the product not only must function for its intended purpose, but it also should feel comfortable to the user. Examples of such cosmetic formulations include foundation, concealer, or other types of cosmetic formulations for application to the face or other areas.

### SUMMARY OF THE DISCLOSURE

In some aspects, the techniques described herein relate to a cosmetic formulation including a water in silicone emulsion for application to an integument, wherein the cosmetic formulation includes: a discontinuous water phase; and a continuous oil phase including: a film former including an MQ resin; an emollient in an amount of 10.0-35.0 wt. % of the formulation; an emulsifier; and a thickener, wherein the formulation subsequent to application to a surface demonstrates wear of at least 24 hours, and wherein the formulation subsequent to application to the surface demonstrates a tack of at least 150 at half an hour subsequent to application to the surface.

In some aspects, the techniques described herein relate to a cosmetic formulation including a water in silicone emulsion for application to an integument, wherein the cosmetic formulation includes: a discontinuous water phase including a first film former and a salt, wherein the first film former is water dispersible; and a continuous oil phase including: a second film former wherein the second film former is oil-dispersible, and an emollient including coconut alkanes and coco-caprylate/caprate, wherein the emollient is provided in an amount of 8.0 to 20.0 wt. % of the formulation, wherein the formulation subsequent to application to a surface demonstrates wear of at least 24 hours.

In some aspects, the techniques described herein relate to a cosmetic formulation including a water in silicone emulsion for application to an integument, wherein the cosmetic formulation includes: a discontinuous water phase; and a continuous oil phase including: a film former including trimethylsiloxysilicate in an amount of about 8.0 to about 15.0 wt. % of the formulation; an emollient including coconut alkanes and coco-caprylate/caprate, the emollient in an amount of about 8.0 to 20.0 wt. % of the formulation; and a gellant including 2 cst Dimethicone (and) Dimethicone/Vinyl Dimethicone Crosspolymer, wherein the formulation subsequent to application to a surface demonstrates wear of at least 24 hours.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 depicts a graph of wear testing results in an example.

FIG. 2 depicts the average tack by minute for sample formulations in an example.

FIG. 3 depicts the average tack of sample formulations in an example.

FIG. 4 depicts viscosity and tack, in addition to playtime, of the sample formulations in an example.

FIG. 5 depicts viscosity and tack of the sample formulations in an example.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The present disclosure describes, among other things, a cosmetic formulation that has good Longwear and is lightweight. The cosmetic formulation can be used, for example, as a foundation. In an example, the cosmetic formulation can be a water in silicone emulsion. The cosmetic formulation can include, for example, both a water dispersible or hydrophilic film former and an oil soluble film former. The cosmetic formulation can additionally include an emollient, and emulsifier, and a mattifying agent.

With conventional cosmetic products, a user typically chooses between a product that has a desired finish and a product that remains on the skin for long periods of time, such as an entire day. Although products may have a desired matte finish, a user may find themselves repeatedly reapplying the product throughout the day, as the product may also be inadvertently removed by sweat and contact with clothing or the like. On the other hand, typical long lasting cosmetic products may have a different sheen or finish than desired. Moreover, Longwear formulations can invertedly feel cakey or heavy, instead of lightweight.

The cosmetic formulation discussed herein can provide for a desired matte finish, long wear, and a lightweight finish in one product. For example, the cosmetic formulation can have a wear of 36 hours, but still provide a fresh, light feeling to the consumer using the cosmetic formulation. Moreover, the cosmetic formulation can have a matte finish. In some cases, the cosmetic formulation can additionally include sun protection.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In the event of inconsistent usages between this document and any documents so incorporated by reference, the usage in this document controls.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the formulation including the material, such that about 0 wt % to about 5 wt % of the formulation is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “wear” as used herein can mean the capacity of a cosmetic formulation for withstanding continuous use without damage or deterioration. The term “longwear” or “long wear” as used herein can mean the capacity for a cosmetic formulation to withstand damage or deterioration for a minimum period of 12 hours.

The term “tack” as used herein can mean the quality of a cosmetic formulation being sticky, such as sticky to the touch, and a measure of how sticky the formulation is when applied to a surface.

### Cosmetic Formulation

According to various aspects of the present disclosure, a cosmetic formulation can deliver desired longwear coverage. The cosmetic formulation can have a desired combination of a film former with a specific emollient system to deliver beneficial properties across a variety of skin types.

The cosmetic formulation can be a water in oil emulsion for application to an integument (e.g., skin of a consumer), such as for use as a foundation. The cosmetic formulation can include a first film former that is water-dispersible and a second film former that is oil soluble. The second film former can be an MQ resin. The cosmetic formulation can additionally include an electrolyte.

The cosmetic formulations discussed herein can be an emulsion. The emulsion can have a viscosity of about 8,000 to 15,000 cP as measured via Brookfield viscosity with a TB spindle at a speed of 10 rpm for one minute. For example, the cosmetic formulation can have a viscosity of about 8,000, 8,500, 9,000, 9,500, 10,000, 10,500, 11,000, 11,500, 12,000, 12,500, 13,000, 13,500, 14,000, 14,500, or about 15,000 cP.

The cosmetic formulation can have, once applied to an integument (e.g., a user's skin or other surface), a Longwear of at least 24 when measured by a consumer panel, or a Longwear of about 24, 25, 26, 27, 28, 29, 30, 31, 32, 33, 34, 35, or about 36 hours.

The cosmetic formulation can have, once applied to an integument (e.g., a user's skin or other surface), a tack of at about 150 to 190 when measured by texture analyzer at 0.5 hours or more, or of about 150, 155, 160, 165, 170, 175, 180, 185, or about 190.

The cosmetic formulation can have, once applied to an integument (e.g., a user's skin or other surface), a Crockmeter dry measurement of about 1.50 to about 2.00, or of about 1.50, 1.55, 1.60, 1.65, 1.70, 1.75, 1.80, 1.85, 1.90, 1.95, or about 2.00.

The cosmetic formulation can have, once applied to an integument (e.g., a user's skin or other surface), a Crockmeter squalene measurement of about 6.00 to about 8.00 or of about 6.00, 6.50, 7.00, 7.50, or about 8.00.

The cosmetic formulation can have, once applied to an integument (e.g., a user's skin or other surface), a Crockmeter water measurement of about 1.00 to about 1.30, or of about 1.00, 1.05, 1.10, 1.15, 1.20, 1.25, or about 1.30.

The cosmetic formulation can have, once applied to an integument (e.g., a user's skin or other surface), a Crockmeter saline measurement of about 1.00 to about 1.40, or of about 1.00, 1.05, 1.10, 1.15, 1.20, 1.25, 1.30, 1.35, or about 1.40.

The cosmetic formulation can have a stability of greater than 1 weeks, or of greater than about 1, 2, 3, 4, 5, 6, 7, 8, 9, 10, 11, 12, 13, 14 or greater than about 15 weeks.

The cosmetic formulation can have an example formulation as summarize in Table 1 below:

TABLE 1
Example cosmetic formulation.

| Phase | Component | Example | Amount |
|---|---|---|---|
| Water Phase | Film former | Polyurethane | 0.0-5.0% |
| | (water phase) | PVP | |
| | Electrolyte | magnesium sulfate | 0.5-1.0% |
| | | sodium chloride | |
| | Water | | 10.0-30.0% |
| Oil Phase | Film former | trimethylsiloxysilicate | 5.0-20.0% |
| | (oil phase) | | |
| | Gellant | 2 cst Dimethicone (and) | 5.0-15.0% |
| | | Dimethicone/Vinyl | |
| | | Dimethicone Crosspolymer | |
| | | 1.5 cst Dimethicone (and) | |
| | | Dimethicone Crosspolymer | |
| | Emollient | C9-12 alkane and Coco- | 10.0-35.0% |
| | | Caprylate/Caprate | |
| | Thickener | Disteardimonium Hectorite | 0.5-2.0% |
| Pigments | Powder | Silica | 5.0-15.0% |
| and Powders | | Titanium dioxide | |
| | Pigments | various | 0.0-8.00% |

| Emulsifiers | cetyl PEG/PPG-10/1 | 3.0-10.0% |
|---|---|---|
| | dimethicone lauryl | |
| | polyglyceryl-3 | |
| | polydimethylsiloxyethyl | |
| | dimethicone | |
| | PEG-10 Dimethicone | |
| | Dimethicone (and) | |
| | Dimethiconol | |

Additional components as desired

Oil Phase. The oil phase of the cosmetic formulation can include a variety of components, including but not limited to film formers, emollients, emulsifiers, vitamins, pigments, thickeners, story ingredients, active components, and other components.

Film formers. The oil phase of the cosmetic formulation can include one or more film formers. The film formers can aid in the formation of a flexible film, and help prevent transfer of the formulation while increasing wear of the formulation. The film formers can include, for example, polymers.

The film formers can include, for example, a silicone MQ resin. An MQ resin is a silicone resin with a unique structure. MQ resins have of mono- (M) and tetra- (Q) functional siloxane units that occupy an important place among organosiloxane polymers. In an example, the MQ resin can be trimethylsiloxysilicate (TMS).

The film former can be provided in an amount of about 5% to about 20% of the formulation, or of about 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20% of the formulation.

The cosmetic formulation can additionally include a water dispersible film former, which can be supplied separately from the oil phase, such as in the water phase. The water dispersible film former can be, for example, polyurethane. The water dispersible film former can be provided in an amount of about 2.5% to about 5.0% of the formulation, or of about 2.5, 3.0, 3.5, 4.0, 4.5, or about 5.0% of the formulation.

The water dispersible film former and the MQ resin film former can be provided at a ratio of about 1:4 to about 1:2. The combination of film formers can allow for better wear and transfer resistance of the cosmetic formulation.

Emollient system. The formulation can include an emollient system, such as to aid in slowing evaporation of water from the formulation.

The emollient system can include, for example, petrolatum, paraffin, mineral oil, glycerin, beeswax, olive oil, coconut oil, lanolin, cocoa butter, synthetics such as butyl stearate and diglycol laurate, dimethicone, diisopropyl sebacate or combinations thereof.

The total amount of emollients in the formulation can be about 10% to about 35%, or of about 10, 11, 12, 13, 14, 15, 16, 17, 18, 19, 20, 21, 22, 23, 24, 25, 26, 27, 28, 29, 30, 31, 32, 33, 34, or 35% of the formulation.

In an example, the emollient can be provided as C9-12 Alkanes and Coco-Caprylate/Caprate.

Emulsifiers. The formulation can include an emulsifier to aid in mixing of the water phase and the oil phase. An emulsifier is particularly suitable when the cosmetic formulation is in the form of an emulsion or if immiscible materials are being combined. The emulsifier can have micellar properties, lamellar properties, or both. Emulsifiers may be nonionic, anionic, zwitterionic, or canonic. The emulsifiers can help provide the lightweight feel of the cosmetic formulation.

In an example, the emulsifier can be about 3.0% to about 8.0% of the formulation, or about 3.5, 4.0, 4.5, 5.0, 5.5, 6.0, 6.5, 7.0, 7.5, or about 8.0% of the formulation. In an example, the emulsifier can include cetyl PEG/PPG-10/1 dimethicone, lauryl polyglyceryl-3 polydimethylsiloxyethyl dimethicone.

Pigments. The formulation can include one or more pigments. The pigments, such as yellow, red, black, brown, or other pigments, can be used for coloration, such as to provide a desired skin shade for the formulation. In some cases pearls or glitters can be used.

The instantly disclosed formulation can include a colorant or pigment component, which can be present in the formulation in a range of from about 5 wt % to about 20 wt % of the formulation, about 8 wt % to about 12 wt % of the formulation, less than, equal to, or greater than about 5 wt %, 10, 15, or 20 wt % of the formulation.

Colorants suitable for use in the present formulations include, but are not limited to, dyes, pigments, lakes, and mixture thereof. (e.g., organic or inorganic pigments and colorants approved for use in face area cosmetics by CTFA and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include FD&C Red No. 40, D&C Green No. 5, FD&C Blue No. 1, and FD&C Yellow No. 5, FD&C Red No. 6, FD&C Red No. 27, FD&C Red No. 28, FD&C Yellow No. 6, and others. Examples of lake dyes include various acid dyes which are laked with aluminum, calcium or barium. Additional colorants for use herein include annatto, caramel, carmine, β-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the CTFA Cosmetic Ingredient Handbook, (1988), published by the Cosmetics, Toiletry and Fragrances Association, Inc. Other colorants may also be used as they are developed and determined safe.

Thickener. The formulation can include one or more thickeners or gellants in the water phase or silicone phase. The thickeners or gellants can aid in the viscosity and tack of the formulation, and aid in coverage.

The thickeners or gellants can include, for example, clay, gum, or cetyl alcohol. In an example, the thickener can include tapioca starch. In an example, the gellant can include crosspolymer dimethicone. In an example, the thickener can include C9-12 alkane, dimethicone and dimethiconal mixtures, or combinations thereof. In an example, a gellant can include 2 cst Dimethicone (and) Dimethicone/Vinyl Dimethicone Crosspolymer; or 1.5 cst Dimethicone (and) Dimethicone Crosspolymer.

In an example, the total amount of thickener or gellant can be about 1.0% to about 15.0% of the formulation, or about 1.0, 2.0, 3.0, 4.0, 5.0, 6.0, 7.0, 8.0, 9.0, 10.0, 11.0, 12.0, 13.0, 14.0, or about 15.0% of the formulation.

In an example, the thickener can include hectorite clay and an activator. The hectorite clay can aid in modifying the viscosity of the formulation and provide a lightweight feel. The hectorite clay can be provided in an amount of about 1.0 to 2.0%. The hectorite can be provided in a solvent and with an activator. The activator can be, for example, propylene carbonate or similar. The activator can be provided in an amount of about 0.3% to about 0.8% of the formulation. The hectorite clay and the activator can be provided in a ratio of about 3:1.

The thickeners and gellants included in the cosmetic formulation can act as mattifying agents to help provide the desired finish and feel of the cosmetic formulation. The thickeners and gellants can additionally help modifying the viscosity of the formulation and provide a lightweight feel.

Solvents. A variety of oil soluble solvents can be used in the oil phase, such as propylene carbonate, isododecane, EDTA, glycerin, or others.

Water Phase. The water phased can include a variety of additional components that are water soluble, such as active ingredients and story ingredients discussed below. A variety of solvents can be in the water phase, such as, for example, water, glycerine, and aqueous solutions such as sodium chloride solution. In an example, the water phase can be about 20% to about 40% of the formulation, or about 20, 21, 22, 23, 24, 25, 26, 27, 28, 29, 30, 31, 32, 33, 34, 35, 36, 37, 38, 39, or about 40% of the formulation. The water phase can help provide a lightweight feel and application of the cosmetic formulation.

Electrolyte components. In an example, the water phased can additionally include one or more electrolyte components, such as magnesium sulfate, sodium chloride, other aqueous salts, or combinations thereof.

Such a rheology modifying component can be about 0.5% to about 1.5% of the formulation, or about 0.5, 0.6, 0.7, 0.8, 0.9. 1.0, 1.1, 1.2, 1.3, 1.4, or about 1.5% of the formulation.

Active Components. A variety of active components can be in the oil phase, the water phase, or both. For example, the formulation can include moisturizing agents, fillers, coloring agents, essence or spice, skin conditioners, vitamins, light protective agents, antiperspirants, antioxidants, crease-resistant materials and other suitable materials.

In an example, the active ingredients can include vitamins, such as vitamin E.

In an example, the active ingredients can include one or more moisturizing agents, such as 2,3-butanediol, Acid, 1-2 Hexanediol, Caprylyl Glycol; Water (and) Propanediol (and) Polyglutamic Acid (and) 1-2 Hexanediol (and) Caprylyl Glycol, or others.

In an example, the active ingredients can include UV protectors, such as zinc oxide, Titanium Dioxide, and others.

In an example, the active ingredients can include fungicidal agents, such as chlorphenesin. In an example, additional active components can include preservatives.

In an example, the active ingredients can include skin conditioning agents, such as dimethicone. In an example, additional components can include triethyl citrate.

Other Components. The formulation can optionally include other components in the oil phase, the water phase, or both. Example additional components can include powders, rheology modifying components, or story ingredients.

For example, absorbent powders such as silica can be included, or other non-absorbent powders can be included. Silica can be included, for example, in an amount of about 5.0% to about 10.0%. Silica can act as a sponge in the formulation. The silica, or other non-absorbent powders, can act as mattifying agents and provide the desired matte finish to the cosmetic formulation. The use of such powders can aid in the overall texture and feel of the cosmetic formulation.

A variety of story ingredients can be included in the formulation, including but not limited to conditioning agents, fibers, butter, functional ingredients, fragrance, and others. In an example, the formulation can include pH modifying ingredients. For example, ethylene brassylate can be used for fragrance.

Other additives may also be included. The formulation can further include an oil-soluble or dispersible additive which may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids).

Further components that can be used are dicarboxylic acids of C₂-C₁₂, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents, hair conditioning agents, vitamins, and the like.

There are any number of further components that can be included in the formulation such as a surfactant component, a skin conditioner component, a humectant component, or another additive component. Other additives can include chamomile and cucumber extract, jojoba oil, camellia oil, aloe butter, maracuja oil, cell-u-lash, marula oil, castor oil, ceramide 2, avocado butter, or a mixture thereof.

Skin Conditioning Agent. The cosmetic formulation can optionally include additional skin conditioning ingredients or agents, such as Propanediol, Glycerin, Nymphaea Caerulea Flower Extract, and Nelumbo Nucifera Flower Extract. For example, such a skin conditioning agent can include extracts from lotus flowers, the Egyptian Lotus (Nymphaea caerulea) and the Sacred Lotus (Nelumbo nucifera). The use of these two extracts together can help synergistically transmit luminosity, reduce inflammation, and reduce sebum production.

In one aspect, the lotus plant derived constituents e.g., Nelupure) may be formulated and/or utilized in products and/or regimens that reduces perspiration in a subject. In some examples, perspiration reduction is accomplished primarily without clogging pores.

In one aspect, the lotus plant derived constituents may be formulated and/or utilized in the form of a primer for creating a substantially flawless canvas for longer lasting makeup following application. In another embodiment, the lotus plant derived constituents may be formulated and/or utilized in the form of a foundation composition. Suitable foundation formulations are those that are capable of evening out skin tone, concealing imperfections and/or providing a flawless matte finish.

In another aspect, the lotus plant derived constituents may be formulated and/or utilized in the form of a cosmetic concealer that substantially conceals small imperfections, dark spots and circles.

In another aspect, the lotus plant derived constituents may be formulated and/or utilized in the form of combination cosmetic foundation/concealer product. In another aspect, the lotus plant derived constituents may be formulated and/or utilized in the form of a setting spray that may be sprayed over make-up to substantially lock in a look (following make-up application) and protect the skin. The lotus plant derived constituents may be utilized to help reduce breakouts caused by make-up and sweat.

### Method of Making Cosmetic Formulation

The instantly described cosmetic formulations can be made according to a variety of suitable methods. As an example, water and the ingredients in the water phase can be added to a beaker and exposed to low shear conditions (about 100 rpm) at room temperatures (about 25 °C). Other components such as pigments, emulsifiers, and the like can be added to create the oil phase. The water phase and the oil phase can be combined with the emulsifier to create the formulation.

### Method of Using

The cosmetic formulation can be contained in an applicator assembly. A variety of conventional applicators are known in the art (e.g., sponges, brushes, wipes, blenders, or other applicators). Optionally, a user can apply the cosmetic formulation by hand. Whether applied by an applicator or by hand, the cosmetic formulation can be applied directly to the skin. The cosmetic formulation can be applied in a single layer, or in multiple layers to create the desired effect. The cosmetic formulation can be blended and shaped by the user on the skin to create the desired coverage.

Optionally, the cosmetic formulation can be used with a variety of other cosmetic products, such as but not limited to skin care compositions, other foundations or concealers, other types of color cosmetic formulations such as blush, bronzer, or highlighter, among others. Each of these can be combined, blended, or adjusted during applicator before, after, or simultaneously with the cosmetic formulation discussed herein, to provide the desired makeup look.

### EXAMPLES

### Example 1. Cosmetic Formulation

For each of the Examples below, a sample cosmetic formulation was produced with the following formulation, except where otherwise noted:

TABLE 2
Sample cosmetic formulation.

| Component | Type | Amount |
|---|---|---|
| Film Former | PVP | 0.0-4.5% |
| (Water Phase) | | |
| Film Former | Trimethylsiloxysilicate | 8.0-12.0% |
| (Oil Phase) | | |
| Electrolyte | Sodium Chloride | 0.2-0.9% |
| Powder | Silica | 5.0-10.0% |
| Gellant | Crosspolymer and dimethicone | 5.0-10.0% |
| Emollients | C9-12 Alkane (and) Coco- | 10.0-25.0% |
| | Caprylate/Caprate | |
| Thickener | hectorite clay | 0.5-2.0% |
| Colorants | zinc oxide, titanium dioxide, and iron | 1.0-25.0% |
| | oxides | |
| Emulsifiers | cetyl PEG/PPG-10/1 dimethicone and | 3.0-15.0% |
| | lauryl polyglyceryl-3 | |
| | polydimethylsiloxyethyl dimethicone | |
| | PEG-10 Dimethicone | |

| | |
|---|---|
| Other components, including water | Remainder |

The following modified samples were produced for testing on Crockmeter (for wear), for tack, and for viscosity:

TABLE 3
Samples for testing, percentages are approximate.

| | | Film | | | | | |
|---|---|---|---|---|---|---|---|
| | | Former | | | | | |
| | Film Former | (Oil | | | | | |
| ID | (Water Phase) | Phase) | Gellant | Salt | Emollient | Emulsifier | Silica |
| LMU 90 | 9.0% Polyurethane | 10.0% | Dowsil EL- | NaCl | 14.1% Vegelight | KF-6105 | SB-700 |
| | Baycusan C1004/1 | BELSIL ® | 9140 (Dow | | C912-LC (Grant | (Shin-Etsu | (Miyoshi |
| | (Covestro) | TMS 803 | Chemical) | | Industries, Inc.) | Silicones | Europe) |
| | | (Wacker) | | | | of America) | |
| LMU | 9.0% Polyurethane | 10.0% | Dowsil EL- | MgSO₄ | 14.1% Vegelight | KF-6105 | SB-700 |
| 106 | Baycusan C1004/1 | BELSIL ® | 9140 (Dow | | C912-LC (Grant | (Shin-Etsu | (Miyoshi |
| | (Covestro) | TMS 803 | Chemical) | | Industries, Inc.) | Silicones | Europe) |
| | | (Wacker) | | | | of America) | |
| LMU | N/A | 19.0% | Dowsil EL- | MgSO₄ | 14.1% Vegelight | KF-6105 | SB-700 |
| 107 | | BELSIL ® | 9140 (Dow | | C912-LC (Grant | (Shin-Etsu | (Miyoshi |
| | | TMS 803 | Chemical) | | Industries, Inc.) | Silicones | Europe) |
| | | (Wacker) | | | | of America) | |
| LMU | 19.0% Polyurethane | N/A | Dowsil EL- | MgSO₄ | 14.1% Vegelight | KF-6105 | SB-700 |
| 108 | Baycusan C1004/1 | | 9140 (Dow | | C912-LC (Grant | (Shin-Etsu | (Miyoshi |
| | (Covestro) | | Chemical) | | Industries, Inc.) | Silicones | Europe) |
| | | | | | | of America) | |
| LMU | 19.0% Micromer | N/A | Dowsil EL- | MgSO₄ | 14.1% Vegelight | KF-6105 | SB-700 |
| 109 | C60/42 (Worlee) | | 9140 (Dow | | C912-LC (Grant | (Shin-Etsu | (Miyoshi |
| | | | Chemical) | | Industries, Inc.) | Silicones | Europe) |
| | | | | | | of America) | |
| LMU | 9.0% BELSIL | 10.0% | SERASILK | MgSO₄ | 10.4% Vegelight | KF-6017 | MSS-500W |
| 131 | TMS 803 | BELSIL ® | EL 65 | | C912-LC (Grant | (Shin-Etsu | (Kobo |
| | (Wacker Chemical | TMS 803 | (KCC | | Industries, Inc.) | Silicones | Products, |
| | Corporation) | (Wacker) | Beauty) | | | of America) | Inc.) |
| FIX 019 | 9.0% BELSIL | 10.0% | SERASILK | MgSO₄ | 10.4% Vegelight | KF-6017 | MSS-500W |
| NOTE: | TMS 803 | BELSIL ® | EL 65 | | C912-LC (Grant | (Shin-Etsu | (Kobo |
| Reduced | (Wacker Chemical | TMS 803 | (KCC | | Industries, Inc.) | Silicones | Products, |
| Pigments | Corporation) | (Wacker) | Beauty) | | | of America) | Inc.) |

LMU 90 was made with the formulation described in Table 2 without modification. LMU 106 was made with the formulation described in Table 2, but replaced the sodium chloride with magnesium sulfate, and increased Bentone. LMU 107, LMU 108, and LMU 109, The result of this testing is discussed below in Examples 2-4.

For LMU 131, the LMU 106 formulation was modified to reduce the amount of Vegelight (the emulsifier) and corresponding increase the solvents; dimethicone/vinyl dimethicone crosspolymer replaced dimethicone crosspolymer as the gellant; the sourced emulsifier and silica were updated as noted in Table 3; and Baycusan was removed and replaced by TMS.

The sample FIX 019 was made as a primer sample, instead of a foundation sample. The sample FIX 019 started with the LMU 131 formulation, but reduced the amounts of yellow and red iron oxide pigments, increased the silica, and increased the dimethicone/vinyl dimethicone crosspolymer. The FIX 019 additionally included 2.0% PVP.

### Example 2. Crockmeter

Samples B to E in Table 3 were tested for wear through the user of a Crockmeter. The results of these tests are summarized below:

TABLE 4
Sample testing results.

| Sample ID | Dry | Squalene | Water | Saline |
|---|---|---|---|---|
| LMU 106 | 1.63 | 6.91 | 1.03 | 1.07 |
| LMU 107 | 1.71 | 7.15 | 1.06 | 1.23 |
| LMU 108 | 4.40 | 15.06 | 1.61 | 1.48 |
| LMU 109 | 3.56 | 13.00 | 3.31 | 4.04 |
| LMU 131 | 1.00 | 2.94 | 1.07 | 1.07 |
| FIX 019 | 0.49 | 1.33 | 1.18 | 1.56 |

These Samples were tested for wear (or “colorfastness”) in a rub test with a Crockmeter under a modified AATCC method 116-2013-Colorfastness to Crocking. Several different rubbing insult types are tested: rub testing as a dry rub, with water, saline, and artificial sebum (squalene). Each of these liquids represents a different liquid that a user might come into contact with throughout the day while wear the formulation. Each liquid (water, saline, squalene) could potentially smear or otherwise transfer the formulation from the user's lashes. Moreover, some of these liquids, such as water, could potentially be used at the end of the day to remove the formulation from the user's lashes. Preferably, the formulation does not smear or transfer under the effects of these liquids, but is also easily removable at the end of the day, such as with the application of water by the user.

For the rub testing, each of the samples was prepared on line strips. One each of the dry, water, saline, and squalene slides were produced with each of the Samples. For the wet samples, a small 20 μL drop of the liquid (water, saline, or squalene) was pipetted onto the sample. The samples were then put into the Crockmeter according to the AATCC method. A manual crank of 10 was used. The rub testing of the samples on the Crockmeter was iterated.

The results of the Crockmeter testing of samples are depicted above in Table 4. A very low Crockmeter value can indicate the sample was difficult to remove, while a very high Crockmeter value can indicate the sample was prone to transfer.

Overall, the formulation LMU 107, with TMS as the second film former and the gel thickener, performed the best across the Crockmeter testing. The dry, water, and saline values for sample LMU 107 were not prone to transfer. By comparison, the LMU 108 and LMU 109 samples with different film formers were more prone to transfer.

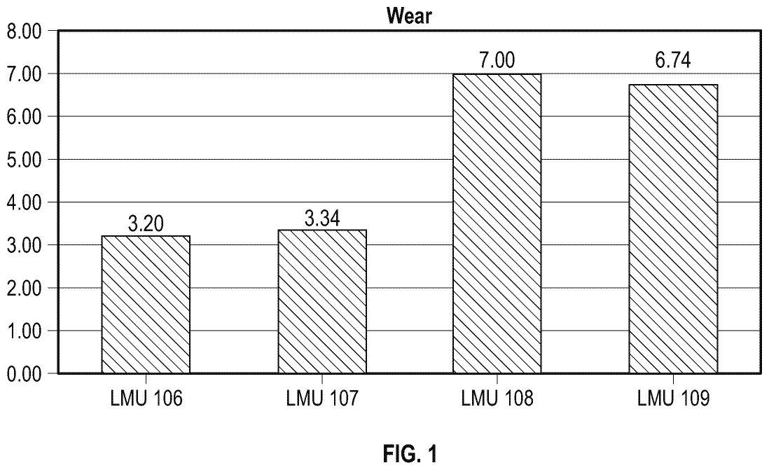

FIG. 1 depicts a graph of the additional wear testing results. Here, transfer of the formulation samples was tested between two cotton squares. A higher value in the graph of FIG. 1 depicts a higher transfer. The samples LMU 108 and LMU 109 with different film formers were very prone to transfer with high assessment values. The LMU 107 sample with the TMS film former was less prone to transfer, showing better wear.

### Example 3. Tack

Samples B to E in Table 3 were tested for. The results of these tests are summarized below:

TABLE 5
Sample testing results.

| Sample ID | Highest Tack | Point |
|---|---|---|
| LMU 106 | 176.06 | 39 |
| LMU 107 | 186.49 | 33 |
| LMU 108 | 211.14 | 37 |
| LMU 109 | 283.91 | 40 |
| LMU 131 | 167.18 | 39 |
| FIX 019 | 193.63 | 29 |

These Samples were tested for tack using a texture analyzer. A steel probe was used on the film over the course of 75 minutes. More specifically, the samples were put on glass plates on top of a fine adjustment hotplate set to 32 degrees Celsius (to emulate skin temperature).

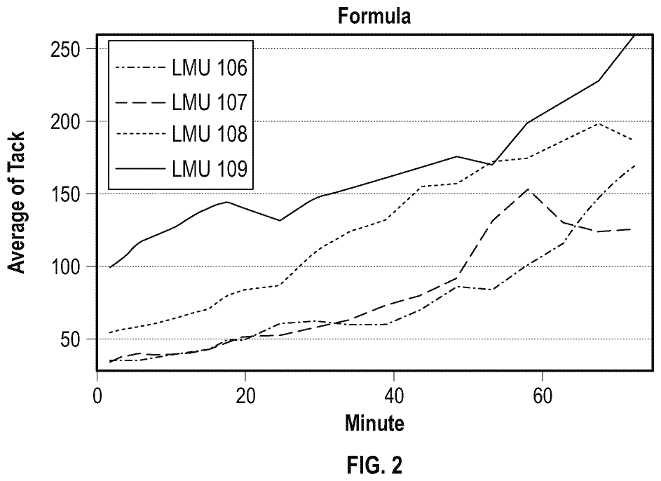

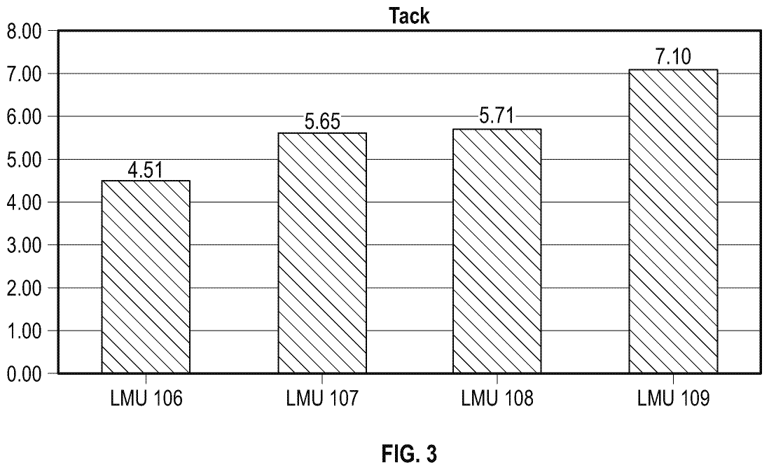

FIGS. 2 and 3 depict graphical results of the tack testing. FIG. 2 depicts the average tack by minute for each sample formulation. FIG. 3 depicts the average tack. Both samples LMU 106 and 107, each containing the TMS film former, performed well, building sufficient tack without going over a desired amount such as about 150. The results of the tack analysis show how tack can build over time.

### Example 4. Viscosity and Playtime

LMU 90 was tested for viscosity. These Samples were tested for viscosity on a Brookfield viscometer with a TB spindle at 10 rpm for one minute. In addition to testing the samples LMU 90, sample of only acrylates, only TMS, only polyurethane, and a combination of acrylates and TMS were tested. The viscosity of the LMU 90 sample were in the range of 8,000 to 10,000 cP.

Viscosity was measured using the Helipath setting on a Brookfield viscometer with a T-spindle (TB) at a fixed rate (10 RPM) for a fixed amount of time (1 minute). Tack was measured with a TA.XT.Plus Texture Analyzer using a 7 mm diameter probe. 100 g of pressure are applied twice to the film every 2.5 minutes for 75 minutes and the pulling force (tackiness) is measured by the instrument. Playtime was extracted by calculating the tackiness over time.

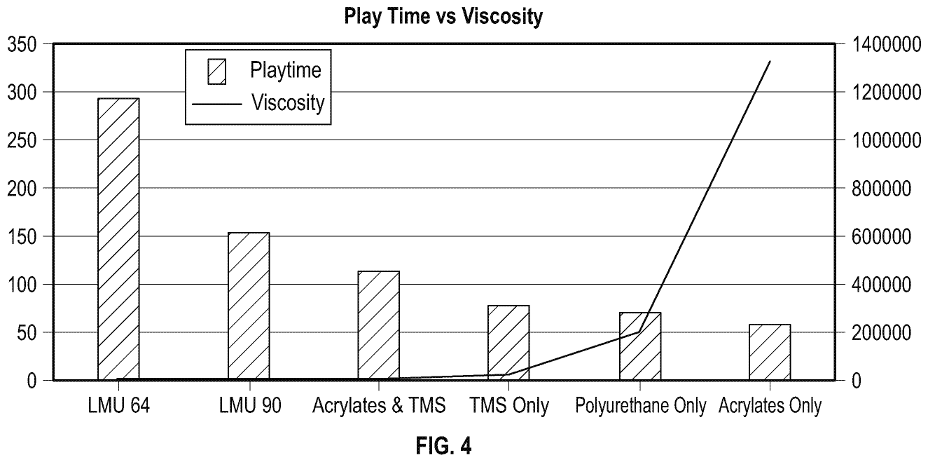

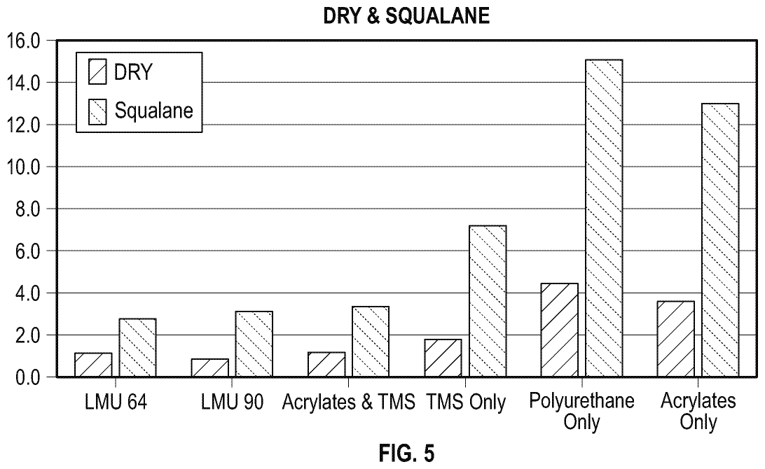

The viscosity and tack results were graphed relative each other. FIGS. 4 and 5 depict both viscosity and tack, in addition to playtime, of the samples.

### Example 5. Stability

Samples using different emulsifier systems will be tested for stability, including sample LMU 106. Comparative samples formulated similar to LMU 106, but with different types of emulsifiers, will also be tested for stability.

In the stability testing, the samples will be kept undisturbed under the same temperature and room conditions for predetermined periods of time. At predetermined intervals, the samples will be reviewed and assessed for stability. The samples will be reviewed visually for separation, texture, and coloration. The samples will also be tested for viscosity and tack as described in the above examples. It is anticipated that the LMU 106 sample, containing the desired emulsifier system, will perform well on stability analysis over time.

### VARIOUS NOTES & EXAMPLES

Example 1. A cosmetic formulation comprising a water in silicone emulsion for application to an integument, wherein the cosmetic formulation comprises: a discontinuous water phase; and a continuous oil phase comprising: a film former including an MQ resin; an emollient in an amount of 10.0-35.0 wt. % of the formulation; an emulsifier; and a thickener, wherein the formulation subsequent to application to a surface demonstrates wear of at least 24 hours, and wherein the formulation subsequent to application to the surface demonstrates a tack of at least 150 at half an hour subsequent to application to the surface.

Example 2. The cosmetic formulation of Example 1, wherein the formulation has a viscosity of 8,000 to 15,000 cP as measured via Brookfield viscosity with a TB spindle at 10 rpm for 1 minute.

Example 3. The cosmetic formulation of Example 1, wherein the formulation comprises a stability of at least 1 week.

Example 4. The cosmetic formulation of Example 1, wherein the formulation subsequent to application to the surface demonstrates Longwear of at least 24 hours when measured by consumer panel.

Example 5. The cosmetic formulation of Example 1, wherein the formulation subsequent to application to surface demonstrates a tack of 150 to 190 at half an hour subsequent to application to the surface.

Example 6. The cosmetic formulation of Example 1, wherein the water phase comprises a hydrophilic film former including water dispersible polymers.

Example 7. The cosmetic formulation of Example 6, wherein the hydrophilic film former comprises 2.5 to 5.0% of the formulation.

Example 8. The cosmetic formulation of Example 6, wherein hydrophilic film former comprises polyurethane.

Example 9. The cosmetic formulation of Example 1, wherein the film former comprises 7.0 to 15.0% of the formulation.

Example 10. The cosmetic formulation of Example 1, wherein film former comprises trimethylsiloxysilicate.

Example 11. The cosmetic formulation of Example 1, wherein the thickener is provided in an amount of 5.0% to 20.0% of the formulation.

Example 12. The cosmetic formulation of Example 1, wherein the emollient comprises C9-12 alkane and coco-caprylate/caprate, dimethicone, dimethiconol, coco-caprylate/caprate, and combinations thereof.

Example 13. The cosmetic formulation of Example 1, further comprising a mattifying agent comprises dimethicone cross polymer, silica, or combinations thereof.

Example 14. A cosmetic formulation comprising a water in silicone emulsion for application to an integument, wherein the cosmetic formulation comprises: a discontinuous water phase comprising a first film former and a salt, wherein the first film former is water dispersible; and a continuous oil phase comprising: a second film former wherein the second film former is oil-dispersible, and an emollient comprising coconut alkanes and coco-caprylate/caprate, wherein the emollient is provided in an amount of 8.0 to 20.0 wt. % of the formulation, wherein the formulation subsequent to application to a surface demonstrates wear of at least 24 hours.

Example 15. The cosmetic formulation of Example 14, further comprising an emulsifier system including cetyl PEG/PPG-10/1 dimethicone and lauryl polyglyceryl-3 polydimethylsiloxyethyl dimethicone.

Example 16. The cosmetic formulation of Example 15, wherein the cetyl PEG/PPG-10/1 dimethicone and lauryl polyglyceryl-3 polydimethylsiloxyethyl dimethicone are provided at a ratio of about 1:1.

Example 17. The cosmetic formulation of Example 14, wherein the first film former comprises polyurethane.

Example 18. The cosmetic formulation of Example 14, wherein the first film former and the second film former are provided at a ratio of 1:4 to 1:2.

Example 19. A cosmetic formulation comprising a water in silicone emulsion for application to an integument, wherein the cosmetic formulation comprises: a discontinuous water phase; and a continuous oil phase comprising: a film former comprising trimethylsiloxysilicate in an amount of about 8.0 to about 15.0 wt. % of the formulation; an emollient comprising coconut alkanes and coco-caprylate/caprate, the emollient in an amount of about 8.0 to 20.0 wt. % of the formulation; and a gellant comprising 2 cst Dimethicone (and) Dimethicone/Vinyl Dimethicone Crosspolymer, wherein the formulation subsequent to application to a surface demonstrates wear of at least 24 hours.

Example 20. The cosmetic formulation of Example 19, wherein the formulation subsequent to application to the surface demonstrates a tack of at least 150 at half an hour subsequent to application to the surface. Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, formulation, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A cosmetic formulation comprising a water in silicone emulsion for application to an integument, wherein the cosmetic formulation comprises:
a discontinuous water phase; and
a continuous oil phase comprising:
a film former including an MQ resin;
an emollient in an amount of 10.0-35.0 wt. % of the formulation;
an emulsifier; and
a thickener,
wherein the formulation subsequent to application to a surface demonstrates wear of at least 24 hours, and
wherein the formulation subsequent to application to the surface demonstrates a tack of at least 150 at half an hour subsequent to application to the surface.

2. The cosmetic formulation of claim 1, wherein the formulation has a viscosity of 8,000 to 15,000 cP as measured via Brookfield viscosity with a TB spindle at 10 rpm for 1 minute.

3. The cosmetic formulation of claim 1, wherein the formulation comprises a stability of at least 1 week.

4. The cosmetic formulation of claim 1, wherein the formulation subsequent to application to the surface demonstrates Longwear of at least 24 hours when measured by consumer panel.

5. The cosmetic formulation of claim 1, wherein the formulation subsequent to application to surface demonstrates a tack of 150 to 190 at half an hour subsequent to application to the surface.

6. The cosmetic formulation of claim 1, wherein the water phase comprises a hydrophilic film former including water dispersible polymers.

7. The cosmetic formulation of claim 6, wherein the hydrophilic film former comprises 2.5 to 5.0% of the formulation.

8. The cosmetic formulation of claim 6, wherein hydrophilic film former comprises polyurethane.

9. The cosmetic formulation of claim 1, wherein the film former comprises 7.0 to 15.0% of the formulation.

10. The cosmetic formulation of claim 1, wherein film former comprises trimethylsiloxysilicate.

11. The cosmetic formulation of claim 1, wherein the thickener is provided in an amount of 5.0% to 20.0% of the formulation.

12. The cosmetic formulation of claim 1, wherein the emollient comprises C9-12 alkane and coco-caprylate/caprate, dimethicone, dimethiconol, coco-caprylate/caprate, and combinations thereof.

13. The cosmetic formulation of claim 1, further comprising a mattifying agent comprises dimethicone cross polymer, silica, or combinations thereof.

14. A cosmetic formulation comprising a water in silicone emulsion for application to an integument, wherein the cosmetic formulation comprises:
a discontinuous water phase comprising a first film former and a salt, wherein the first film former is water dispersible; and
a continuous oil phase comprising:
a second film former wherein the second film former is oil-dispersible, and
an emollient comprising coconut alkanes and coco-caprylate/caprate, wherein the emollient is provided in an amount of 8.0 to 20.0 wt. % of the formulation,
wherein the formulation subsequent to application to a surface demonstrates wear of at least 24 hours.

15. The cosmetic formulation of claim 14, further comprising an emulsifier system including cetyl PEG/PPG-10/1 dimethicone and lauryl polyglyceryl-3 polydimethylsiloxyethyl dimethicone.

16. The cosmetic formulation of claim 15, wherein the cetyl PEG/PPG-10/1 dimethicone and lauryl polyglyceryl-3 polydimethylsiloxyethyl dimethicone are provided at a ratio of about 1:1.

17. The cosmetic formulation of claim 14, wherein the first film former comprises polyurethane.

18. The cosmetic formulation of claim 14, wherein the first film former and the second film former are provided at a ratio of 1:4 to 1:2.

19. A cosmetic formulation comprising a water in silicone emulsion for application to an integument, wherein the cosmetic formulation comprises:
a discontinuous water phase; and
a continuous oil phase comprising:
a film former comprising trimethylsiloxysilicate in an amount of about 8.0 to about 15.0 wt. % of the formulation;
an emollient comprising coconut alkanes and coco-caprylate/caprate, the emollient in an amount of about 8.0 to 20.0 wt. % of the formulation; and
a gellant comprising 2 cst Dimethicone (and) Dimethicone/Vinyl Dimethicone Crosspolymer,
wherein the formulation subsequent to application to a surface demonstrates wear of at least 24 hours.

20. The cosmetic formulation of claim 19, wherein the formulation subsequent to application to the surface demonstrates a tack of at least 150 at half an hour subsequent to application to the surface.
